(19) United States
(12) Patent Application Publication
(10) Pub. No.: US 2025/0042831 A1
(43) Pub. Date: Feb. 6, 2025

(54) PROCESS FOR PREPARING 5-CHLORO-2, 3-DIHYDRO-1H-INDEN-1-ONE

(71) Applicants: ADAMA MAKHTESHIM LTD., Beer Sheva (IL); NANKAI UNIVERSITY, Tianjin (CN)

(72) Inventors: Wei LI, Tianjin (CN); Yu ZHAO, Tianjin (CN); Dingwen MIAO, Tianjin (CN); Mannam SREEDEVI, Hyderabad (IN); Bijukumar Gopinathan PILLAI, Hyderabad (IN)

(73) Assignees: ADAMA MAKHTESHIM LTD., Beer Sheva (IL); NANKAI UNIVERSITY, Tianjin (CN)

(21) Appl. No.: 18/717,292

(22) PCT Filed: Dec. 7, 2021
(86) PCT No.: PCT/CN2021/136017

§ 371 Date: Jun. 6, 2024

(51) Int. Cl. C07C 49/697

(52) U.S. Cl. CPC C07C 49/697; C07C 2529/40

(57) ABSTRACT

The present disclosure provides a process for preparing 5-chloro-2,3-dihydro-1H-inden-1-one and the 5-chloro-2,3-dihydro-1H-inden-1-one prepared by the process. The present disclosure also provides a process for preparing Indoxacarb and the Indoxacarb prepared by the process.

### TECHNICAL FIELD OF THE DISCLOSURE

The present disclosure relates to a process for preparing 5-chloro-2,3-dihydro-1H-inden-1-one and the 5-chloro-2,3-dihydro-1H-inden-1-one prepared by the process. The presents disclosure also relates to a process for preparing Indoxacarb and the Indoxacarb prepared by the process.

### FIELD AND BACKGROUND OF THE DISCLOSURE

5-chloro-2,3-dihydro-1H-inden-1-one, abbreviated as 5-chloroindanone, is an important intermediate for e.g., oxadiazines agrochemical compounds like Indoxacarb and some other pharmaceutical compounds.

U.S. Pat. No. 5,811,585A discloses a process for preparing 5-chloro-2,3-dihydro-1H-inden-1-one which comprises contacting 3-chloro-1-(4-chlorophenyl)-1-propanone with a catalyst selected from sulfuric acid and solid acid catalysts having a silicon to aluminum ratio of 2.0 to 150. This document specifies the solid acid catalysts as zeolites, especially HZSM-5, HZSM-11, H-Mordenite, H-Y, and H-Beta. This document also mentions when a solid acid catalyst is used, the reaction is preferably carried out in a continuous flow fixed-bed reactor system using an inert carrier gas. Except those, however, nowhere in this document mentions the carrier gas again, let alone the effect of the carrier gas or the flow rate of the carrier gas. This document is also silent on the importance of selecting a suitable flow rate of the carrier gas to the process, especially the yield, the conversion, or the selectivity.

### SUMMARY OF THE DISCLOSURE

Surprisingly, it has now been found that, the flow rate of the carrier gas is very important for preparing 5-chloro-2,3-dihydro-1H-inden-1-one in a continuous way. More specifically, it has now been found that, the flow rate of the carrier gas should be carefully selected to optimize the yield, the conversion, and/or the selectivity.

The present disclosure provides a process for preparing 5-chloro-2,3-dihydro-1H-inden-1-one and the 5-chloro-2,3-dihydro-1H-inden-1-one prepared by the process. The presents disclosure also provides a process for preparing Indoxacarb and the Indoxacarb prepared by the process.

Specifically, the present disclosure provides:
1. A process for preparing 5-chloro-2,3-dihydro-1H-inden-1-one comprising contacting 3-chloro-1-(4-chlorophenyl)-1-propanone continuously with a catalyst in the presence of an inert carrier gas at a gas hourly space velocity (GHSV) of 0.5 to 50 milliliter per minute per gram of catalyst.
2. The process of embodiment 1, wherein the GHSV of the inert carrier gas is 1 to 40 milliliter per minute per gram of catalyst.
3. The process of embodiment 1, wherein the GHSV of the inert carrier gas is 2 to 35 milliliter per minute per gram of catalyst.
4. The process of embodiment 1, wherein the GHSV of the inert carrier gas is 5 to 30 milliliter per minute per gram of catalyst.
5. The process of embodiment 1, wherein the GHSV of the inert carrier gas is 9 to 28 milliliter per minute per gram of catalyst.
6. The process of embodiment 1, wherein the GHSV of the inert carrier gas is 10 to 15 milliliter per minute per gram of catalyst.
7. The process of embodiment 1, wherein the catalyst is selected from the group consisting of a zeolite catalyst, MCM-41, and the combination thereof.
8. The process of embodiment 7, wherein the zeolite catalyst is selected from the group consisting of HY, Hβ, H-Mordenite, HZSM, and a combination thereof.
9. The process of embodiment 8, wherein the HZSM is HZSM-5 with a silicon to aluminum ratio of 15 to 300.
10. The process of embodiment 9, wherein the catalyst is HZSM-5 with a silicon to aluminum ratio of 25 to 200.
11. The process of any one of embodiments 8 to 10, wherein the catalyst has been modified by calcine in air at a temperature of 400 to 1000° C.
12. The process of embodiment 11, wherein the catalyst has been further modified by a steam.
13. The process of embodiment 12, wherein the steam modification is conducted in the presence of an inert carrier gas.
14. the process of embodiment 1 or 13, wherein the inert carrier gas is nitrogen gas.
15. The process of any one of embodiments 12-14, wherein the steam modification is carried out at a temperature of 400 to 900° C.
16. The process of any one of embodiments 11 to 15, wherein the catalyst has been further modified by doping with an element selected from the group consisting of P, B, Zn, Ni, Mg, Cu, and Fe.
17. The process of any one of embodiments 1 to 16, wherein the 3-chloro-1-(4-chlorophenyl)-1-propanone is fed to the catalyst as a solution in an inert solvent.
18. The process of embodiment 17, wherein the solvent is 1,2,3,4-tetrahydronaphthalene.
19. The process of embodiment 17 or 18, wherein the flow rate of 3-chloro-1-(4-chlorophenyl)-1-propanone is 0.5 to 10 g per g catalyst per hour.
20. The process of any one of embodiments 1 to 19, wherein the contacting is carried out in a continuous flow reactor.
21. The 5-chloro-2,3-dihydro-1H-inden-1-one prepared by the process according to any one of embodiments 1 to 20.
22. A process for preparing Indoxacarb comprising the steps of:
1) preparing 5-chloro-2,3-dihydro-1H-inden-1-one by the process according to any one of embodiments 1 to 20; and
2) preparing Indoxacarb from 5-chloro-2,3-dihydro-1H-inden-1-one prepared in step 1).
23. The Indoxacarb prepared by the process according to embodiment 22.

### BRIEF DESCRIPTION OF THE DRAWINGS

FIG. 1 shows schematically the continuous flow fixed-bed reactor used in the Examples.

In FIG. 1, the reference signs are listed as follows:
1. Nitrogen cylinder
2. The reactant solution
3. Peristaltic pump
4. Flowmeter
5. Inert packing material
6. Catalyst bed
7. Condenser
8. GC (Gas chromatography)

FIG. 2 shows the reaction data at a nitrogen gas hourly space velocity of 12.5 milliliter per minute per gram of catalyst in the Example 4.

FIG. 3 shows the reaction data at a nitrogen gas hourly space velocity of 18.75 milliliter per minute per gram of catalyst in the Example 4.

FIG. 4 shows the reaction data at a nitrogen gas hourly space velocity of 25 milliliter per minute per gram of catalyst in the Example 4.

### DESCRIPTION OF SPECIFIC EMBODIMENTS OF THE DISCLOSURE

### Definitions

Prior to setting forth the present subject matter in detail, it may be helpful to provide definitions of certain terms to be used herein. Unless defined otherwise, all technical and scientific terms used herein have the same meaning as is commonly understood by one of skill in the art to which this disclosure pertains.

Indoxacarb is the common name for (S)-methyl 7-chloro-2,5-dihydro-2-[[(methoxycarbonyl)[4-(trifluoromethoxy)phenyljamino]carbonyllindeno[1,2-e][1,3,4]oxadiazine-4a(3H)-carboxylate with a structure of

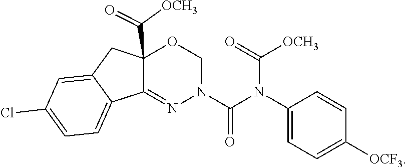 [SMILES: COC(=O)N(C(=O)N1CO[C@@]2(C(=O)OC)Cc3cc(Cl)ccc3C2=N1)c1ccc(OC(F)(F)F)cc1]

As used herein, the term Gas Hourly Space Velocity or “GHSV” means the 5 unit volume of gas at normal temperature and pressure (0° C., 1 atm, i.e. 101.3 kPa) passing over one unit weight of packed catalyst per minute.

As used herein, the term “conversion” means efficiency of raw material conversion per unit time, which is calculated according to the following equation:

$$Conversion = ( {S}_{0} - {S}_{1} ) / {S}_{0}$$

S₀ represents the peak area of all compounds in the reaction solution (except solvent peak).
S₁ represents the peak area of the reactant in the reaction solution, that is, 3-chloro-1-(4-chlorophenyl)-1-propanone.

As used herein, the term “selectivity” means proportion of target products in all products, which is calculated according to the following equation:

$$Selectivity = {S}_{2} / ( {S}_{0} - {S}_{1} )$$

S₀ represents the peak area of all compounds in the reaction solution (except solvent peak),
S₁ represents the peak area of the reactant in the reaction solution, that is, 3-chloro-1-(4-chlorophenyl)-1-propanone,
S₂ represents the peak area of the product in the reaction solution, that is, 5-chloro-2,3-dihydro-1H-inden-1-one,

As used herein, the term “yield” means theoretical yield obtained from chromatographic data after reaction, which is calculated according to the following equation:

$$Yield = {S}_{2} / {S}_{0} OR Yield = Conversion \times Selectivity$$

S₀ represents the peak area of all compounds in the reaction solution (except solvent peak),
S₂ represents the peak area of the product in the reaction solution, that is, 5-chloro-2,3-dihydro-1H-inden-1-one.

The term “a” or “an” as used herein includes the singular and the plural, unless specifically stated otherwise. Therefore, the term “a,” “an,” or “at least one” can be used interchangeably in this application.

It is understood that where a parameter range is provided, all integers within that range, and tenths thereof, are also provided by the disclosure as if the integers and tenths thereof are expressly described herein. For example, “0.1% to 70%” includes 0.1%, 0.2%, 0.3%, 0.4%, 0.5% etc. up to 70%.

Throughout the application, descriptions of various embodiments use the term “comprising”; however, it will be understood by one of skill in the art, that in some specific instances, an embodiment can alternatively be described using the language “consisting essentially of” or “consisting of”.

For purposes of better understanding the present teachings and in no way limiting the scope of the teachings, unless otherwise indicated, all numbers expressing quantities, percentages, or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques. In this regard, use of the term “about” herein specifically includes ±10% from the indicated values in the range. In addition, the endpoints of all ranges directed to the same component or property herein are inclusive of the endpoints, are independently combinable, and include all intermediate points and ranges.

### First Aspect

In the first aspect, the present disclosure provides a process for preparing 5-chloro-2,3-dihydro-1H-inden-1-one. In the first aspect, the present disclosure also provides the 5-chloro-2,3-dihydro-1H-inden-1-one prepared by the process.

The process for preparing 5-chloro-2,3-dihydro-1H-inden-1-one comprises contacting 3-chloro-1-(4-chlorophenyl)-1-propanone continuously with a catalyst in the presence of an inert carrier gas at a gas hourly space velocity (GHSV) of 0.5 to 50 milliliter per minute per gram of catalyst.

In the process of the first aspect, the inert carrier gas may be any inert gases, for example, Ar, He, N₂, etc. The inert carrier gas is preferably N₂.

In the process, the GHSV of the inert carrier gas may be 0.5 to 50 milliliter per minute per gram of catalyst (ml/(min·g cat.)). Within the range of 0.5 to 50 ml/(min·g cat.), the GHSV of the inert carrier gas may be 0.5, 0.6, 0.7, 0.8, 0.9, 1, 2, 2.5, 3, 4, 5, 6, 7, 8, 9, 10, 11, 12, 12.5, 13, 14, 15, 16, 17, 18, 18.75, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 ml/(min·g cat.), or any ranges formed by selecting any two GHSVs mentioned above as the two limits, for example, the GHSV of the inert carrier gas may be 0.5 to 40, or 5 to 25 ml/(min·g cat.). Preferably, the GHSV of the inert carrier gas may be 1 to 40 ml/(min·g cat.). More preferably, the GHSV of the inert carrier gas may be 2 to 35 ml/(min·g cat.). More preferably, the GHSV of the inert carrier gas may be 5 to 30 ml/(min·g cat.). More preferably, the GHSV of the inert carrier gas may be 9 to 28 ml/(min·g cat.). Most preferably, the GHSV of the inert carrier gas may be 10 to 15 ml/(min·g cat.). For example, the GHSV of the inert carrier gas may be 12.5 ml/(min·g cat.). It has now been found that, in order to optimize the yield, the conversion, and/or the selectivity of the process for preparing 5-chloro-2,3-dihydro-1H-inden-1-one in a continuous mode, the GHSV of the carrier gas should be carefully selected.

In the process of the first aspect, the catalyst may be conventional solid acid catalysts, e.g., those mentioned in U.S. Pat. No. 5,811,585A. Preferably, the catalyst may be selected from the group consisting of a zeolite catalyst, MCM-41, and the combination thereof.

Zeolites are complex aluminosilicates that comprise SiO₄ and AlO₄ tetrahedral linked at their corners via common oxygen atoms. It is well known in the art that the small cations within the zeolites can be removed by ion-exchange with NH₄⁺ ammonium cations, then the ammonium ion exchanged zeolites can be thermolyzed to liberate ammonia, leaving behind sites on the catalyst framework comprising Bronstead acidic H⁺ cations attached to oxygen atoms in the framework, thus forming zeolites that are solid acids. The zeolite catalyst which may be used in the present disclosure are solid acids. Suitable zeolite catalyst which may be used in the present disclosure may be selected from the group consisting of HY, Hβ, H-Mordenite, HZSM-5, HZSM-11, and a combination thereof. Preferably, the zeolite catalyst which may be used in the present disclosure may be selected from the group consisting of HY, Hβ, HZSM-5, and a combination thereof. The zeolite catalyst can be characterized by the Si to Al ratio of their framework. In some embodiments, the Si/Al ratio is from 5 to 500. Within the range of 2 to 500, the Si/Al ratio may be 2, 3, 4, 5, 10, 15, 20, 25, 30, 35, 36, 40, 50, 60, 70, 80, 90, 100, 110, 120, 130, 140, 150, 160, 170, 180, 190, 200, 250, 300, 350, 400, 450, 500, or any ranges formed by selecting any two ratios mentioned above as the two limits, for example, the Si/Al ratio may be 15 to 400, or 10 to 450. For HY, the Si/Al ratio may be 2 to 200, preferably 4 to 100, more preferably 5 to 20. For Hβ, the Si/Al ratio may be 5 to 100, preferably 10 to 50, more preferably 10 to 30. For HZSM-5, the Si/Al ratio may be 15 to 300, preferably 20 to 250, more preferably 25 to 200.

MCM-41 catalyst is a nanostructure material, which has the characteristics of hexagonal ordered arrangement, uniform size, continuous adjustment of pore size in the range of 2-10 nm, large specific surface area and so on.

In the process of the first aspect, when the catalyst is a zeolite catalyst e.g., those selected from the group consisting of HY, Hβ, H-Mordenite, HZSM, and a combination thereof, the zeolite catalyst may have been modified by calcine in air at a temperature of 400 to 1000° C., preferably at 550 to 900° C. The heating rate is generally 2-20° C./min. The duration for calcine modification is generally 2-10 hours. Calcine modification on the zeolite catalyst is conventional to those skilled in the art. Calcine modification generally will remove any impurities in the zeolite catalyst and lead to skeleton dealumination of molecular sieve and the formation of new coordinated aluminum on the surface.

In some embodiments, in addition to calcine modification, the zeolite catalyst may have been further modified by a steam. The steam modification may be conducted in the presence of an inert carrier gas. The inert carrier gas may be any inert gases, for example, Ar, He, N₂, etc. The inert carrier gas is preferably N₂. The inert carrier gas used for steam modification may be the same as or different from that used for preparing 5-chloro-2,3-dihydro-1H-inden-1-one mentioned before. The GHSV of the inert carrier gas may be 10-90 milliliter per minute per gram of catalyst (ml/(min·g cat.)), preferably 40-60 ml/(min·g cat.). The steam modification may be carried out at a temperature of 400 to 900° C., preferably 500 to 800° C. The heating rate is generally 2-20° C./min. The duration for steam modification is generally 2-10 hours. The steam modification at high temperature is generally used to modify the acidity of the zeolite catalyst.

In some embodiments, in addition to or independent from the steam modification, the zeolite catalyst may have further been modified by doping with an element selected from the group consisting of P, B, Zn, Ni, Mg, Cu, and Fe. The doping may be carried out by any conventional means in the art, for example, impregnation, ion exchange, chemical deposition, etc. The element may be doped in a form of acid, a salt, etc. For example, element P may be doped in a form of (NH₄)₂HPO₃, NH₄H₂PO₃, or H₃PO₃. For another example, element B, Zn, Ni, Mg, Cu and Fe may be doped in a form of H₃BO₃, Zn(NO₃)₂, Ni(NO₃)₂, Mg(NO₃)₂, ZnCl₂, CuCl₂, or FeCl₂, respectively. The doping amount of the element may be conventional in the art. For example, the doping amount of an element is generally 0.05 to 10 percent by weight based on the total weight of the catalyst. Preferably, the doping amount of an element is 1 to 5 percent by weight based on the total weight of the catalyst.

The amount of the catalyst is generally depending on the flow rate of the raw material 3-chloro-1-(4-chlorophenyl)-1-propanone, which is described later.

In some embodiments, the zeolite catalyst is those doped with an element selected from the group consisting of P, B, Zn, Ni, Mg, Cu, and Fe as mentioned before. Before used in the process of the first aspect, the doped catalyst has been modified by calcine in air in the same way as mentioned before. In addition to or independent from calcine modification, the doped catalyst may have been further modified by a steam in the same way as mentioned before.

In the process of the first aspect, the 3-chloro-1-(4-chlorophenyl)-1-propanone may be fed to the catalyst as a solution in an inert solvent. The inert solvent may be any conventional inert solvents for 3-chloro-1-(4-chlorophenyl)-1-propanone such as 1,2-dichlorobenzene, chlorobenzene, 1,1,2,2-tetrachloroethylene, 1,2,3,4-tetrahydronaphthalene, decahydronaphthalene, nitrobenzene, xylenes, or a combination thereof. Preferred solvents include 1,2-dichlorobenzene, chlorobenzene, tetrachloroethylene, 1,2,3,4-tetrahydronaphthalene, decahydronaphthalene, xylenes, or a combination thereof. Most preferably, the solvent is 1,2,3,4-tetrahydronaphthalene. When a solvent is employed, the concentration of the reactant 3-chloro-1-(4-chlorophenyl)-1-propanone in the solvent is not limited except by its solubility in the particular solvent selected. In general, the concentration of the reactant 3-chloro-1-(4-chlorophenyl)-1-propanone in a solvent is 1 to 99 percent by weight based on the total weight of the solvent and the reactant. For example, the concentration of the reactant 3-chloro-1-(4-chlorophenyl)-1-propanone in a solvent may be 2 to 50, 3 to 25, 4 to 20, to 15, or 6 to 13, or 10 percent by weight based on the total weight of the solvent and the reactant.

In the process of the first aspect, any suitable flow rate of 3-chloro-1-(4-chlorophenyl)-1-propanone per gram of catalyst per hour may be employed. In general, a flow rate of between 0.1 and 20 g of 3-chloro-1-(4-chlorophenyl)-1-propanone per gram of catalyst per hour is employed. Preferably, a flow rate of between 0.5 and 10 g of 3-chloro-1-(4-chlorophenyl)-1-propanone per gram of catalyst per hour is employed. More preferably, a flow rate of between 1 and 5 g (for example, 2.5 g) of 3-chloro-1-(4-chlorophenyl)-1-propanone per gram of catalyst per hour is employed. Lower flow rates are less practical while higher flow rates may result in low conversion to 5-chloro-2,3-dihydro-1H-inden-1-one.

In the process of the first aspect, 3-chloro-1-(4-chlorophenyl)-1-propanone contacts with a catalyst in a continuous way. In some embodiments, a continuous flow reactor, for example, a fix-bed continuous flow reactor, is used in the process for contacting 3-chloro-1-(4-chlorophenyl)-1-propanone with a catalyst. A continuous flow reactor, for example, a fix-bed continuous flow reactor is conventional in the art. FIG. 1 shows schematically a continuous flow fixed-bed reactor system used in the Examples of the present disclosure.

In FIG. 1, a nitrogen cylinder 1 is used to provide the driving force, and a flowmeter 4 is used to control the flow rate of nitrogen. The reactant is dissolved in appropriate solvent to form a solution 2, and the solution 2 is pumped by a peristaltic pump 3 at a constant flow rate into a stainless steel tube reactor, which is for example 16 mm internal diameter, and 70 cm long. The tube of the reactor is divided to a preheat zone and a constant temperature zone, as known by those skilled in the art.

The upper part of the tube is filled with an inert packing material 5 (e.g., ceramic rings) to provide better heat transfer to the incoming feed stream. Heating of the reactor is achieved by enclosing it in a tube furnace with refractory embedded heating elements which maintained uniform temperature across the reaction zone, which is filled with the corresponding catalyst. The reactor temperature is metered and monitored by thermocouples (not shown) embedded in the midpoint of the catalyst bed 6 and the external wall of the reactor. The product stream or effluent from the reactor is directed to a trapping system, comprised of a cooling circulating pump (not shown) and a condenser 7. The volatile organics are condensed and collected for analysis.

Any uncondensed vapors are directed to a scrubber (not shown). The inert unscrubbed gas is vented to the atmosphere. The analysis of the product is achieved using for example gas-chromatography 8.

The inventors surprisingly found that the flow rate of the carrier gas will significantly impact the process for preparing 5-chloro-2,3-dihydro-1H-inden-1-one, especially the yield, the conversion, and/or the selectivity. The inventors surprisingly found that by selecting a suitable gas hourly space velocity of the inert carrier gas, both the conversion of the raw material to other byproducts and the conversion of the desired final product to other byproducts can be suppressed. By suitably controlling the gas hourly space velocity of the inert carrier gas, the process in the first aspect can prepare 5-chloro-2,3-dihydro-1H-inden-1-one in a high conversion, a high selectivity and/or a high yield, or a balance of conversion, selectivity and yield.

The first aspect also provides the 5-chloro-2,3-dihydro-1H-inden-1-one prepared by the process as described in the first aspect. 5-chloro-2,3-dihydro-1H-inden-1-one is an important intermediate for e.g., oxadiazines agrochemical compounds like Indoxacarb and some other pharmaceutical compounds.

### Second Aspect

In the second aspect, the present disclosure provides a process for preparing Indoxacarb and the Indoxacarb prepared by the process of the second aspect.

The process for preparing Indoxacarb comprises the steps of:
1) preparing 5-chloro-2,3-dihydro-1H-inden-1-one by the process as described in the first aspect; and
2) preparing Indoxacarb from 5-chloro-2,3-dihydro-1H-inden-1-one prepared in step 1).

In step 1) of the process, 5-chloro-2,3-dihydro-1H-inden-1-one is prepared by the process as described in the first aspect. Therefore, all the specific descriptions made on the process for preparing 5-chloro-2,3-dihydro-1H-inden-1-one in the first aspects apply to the step 1) as all relevant descriptions have been copied here. For example, unless otherwise indicated, all the specific descriptions on contacting, inert carrier gas, GHSV, catalyst comprising calcining, steam modification and doping, reactor, relevant materials used therein (e.g., 3-chloro-1-(4-chlorophenyl)-1-propanone, solvents, etc.), conditions (e.g., temperature and time, etc.), and the like specified in the first aspect apply to here in the second aspect as all relevant specific descriptions have been copied here.

In step 2) of the process, Indoxacarb is prepared from 5-chloro-2,3-dihydro-1H-inden-1-one prepared in step 1). Preparing Indoxacarb from 5-chloro-2,3-dihydro-1H-inden-1-one is known in the art. For example, step 2) preparing Indoxacarb from 5-chloro-2,3-dihydro-1H-inden-1-one may comprise preparing 5-chloro-1-oxo-2,3-indan-2-carboxylic acid methyl ester from 5-chloro-2,3-dihydro-1H-inden-1-one, preparing (+)-5-chloro-2,3-dihydro-2-hydroxy-1-oxo-2H-indene-2-carboxylic acid methyl ester from 5-chloro-1-oxo-2,3-indan-2-carboxylic acid methyl ester, reaction with benzyl carbazate, cyclization, deprotection, condensation, and the like.

In the second aspect, the present disclosure also provides the Indoxacarb prepared by the process of the second aspect. Indoxacarb is an insecticide for some fruits and vegetables.

Each embodiment disclosed herein is contemplated as being applicable to each of the other disclosed embodiments. Thus, all combinations of the various elements described herein are within the scope of the disclosure. In addition, the elements recited in process embodiments can be used in combination with compound embodiments described herein and vice versa.

This disclosure will be better understood by reference to the Examples which follow, but those skilled in the art will readily appreciate that the specific experiments detailed are only illustrative of the disclosure as described more fully in the claims which follow thereafter.

The disclosure is illustrated by the following examples without limiting it thereby.

### EXAMPLES

### General Treatment Procedures for Catalysts

Calcination modification: A catalyst is calcined in air by heating to a predetermined temperature between 500 to 900° C. at a rate of 10° C./hr and holding at the temperature for 5 hr. It is then allowed to cool down to room temperature. Then the catalyst is tableted and screened, and the catalyst with particle size of 20-60 mesh is selected for subsequent treatment or experiments.

Steam modification: 20-60 mesh catalyst particles are selected and placed in a vertical furnace. The catalyst is calcined in nitrogen atmosphere by heating to a predetermined temperature between 500 to 900° C. at a rate of 10° C./hr. Then nitrogen is introduced into a three-port flask containing water with a heating temperature of 100° C. to drive water vapor into the tubular furnace. The hydrothermal treatment is maintained for 5 h at the same temperature. Then water vapor injecting is stopped, and nitrogen is directly introduced into the tubular furnace. The tubular furnace is cooled to room temperature for later use.

### General Configuration for Reactor System

In the continuous flow fixed-bed reactor system as shown in FIG. 1 with a 16 mm 1.D., and 70 cm long stainless steel tube fittings to connect the feed delivery unit, product recovery unit and a thermo-couple, the modified catalyst is packed in the stainless steel tube, and the catalyst bed is preceded and followed by layers of inert packing materials (ceramic rings) to provide better heat transfer to the incoming feed stream.

Heating of the reactor is achieved by enclosing it in a tube furnace with refractory embedded heating elements which maintained uniform temperature across the reaction zone. The reactor temperature is metered and monitored by thermocouples embedded in the midpoint of the catalyst bed. The reactor feed system is designed to allow vapor and liquid feeds into the reaction zone at a constant flow rate.

General Procedures for Preparing 5-chloro-2,3-dihydro-1H-inden-1-one

A solution 2 of 3-chloro-1-(4-chlorophenyl)-1-propanone in a solvent is continuously pumped into the tube reactor as shown in FIG. 1 by peristaltic pump 3 at a predetermined constant flow rate and contacts continuously with the catalyst in the tube.

Meantime, nitrogen gas is also introduced from the nitrogen cylinder 1 into the tube reactor at a predetermined constant flow rate to provide a driving force to the reactant solution and also the reaction product. Nitrogen gas flow is metered and monitored using a flow meter 4.

The product stream or effluent from the reactor is directed to a trapping system comprised of an ice-cooled trap. The volatile organics are condensed and collected for work up and analysis. Any uncondensed vapors are directed to two scrubbers, set in series. The inert unscrubbed gases are vented to the atmosphere. Product identification and quantitation are achieved using one or more of the following techniques: gas-chromatography, mass spectrometry and nuclear magnetic resonance.

### Example 1

A 10% by weight 3-chloro-1-(4-chlorophenyl)-1-propanone solution 2 in 1,2,3,4-tetrahydronaphthalene (THN) was introduced continuously into the tube reactor as shown in FIG. 1 as configured above but without the catalyst. Meanwhile, nitrogen gas was pumped continuously into the tube at the same time. The feed rate of the solution 2 was adjusted to 10 g/h by peristaltic pump 3, the reaction temperature was 350° C., and the nitrogen flow rate was controlled to 10 mL/min, 30 mL/min, 50 mL/min., and 70 mL/min. The products at different reaction time were collected and the corresponding chromatographic data (peak area) was obtained using Agilent 7890A.

TABLE 1
Results at a 10 mL/min N₂

| Reaction Time(h) | 0 | 1 | 2 | 3 | 4 | 5 |
|---|---|---|---|---|---|---|
| Peak area of solvent(%) | 91.47 | 91.76 | 92.37 | 92.46 | 92.48 | 92.39 |
| Peak area of p- | 0.00 | 0.30 | 0.29 | 0.28 | 0.28 | 0.27 |
| chloroacetophenone(%) | | | | | | |
| Peak area of p- | 0.00 | 0.54 | 0.69 | 0.65 | 0.69 | 0.63 |
| chloropropiophenone(%) | | | | | | |
| Peak area of raw material(%) | 4.65 | 1.87 | 2.32 | 2.36 | 2.36 | 2.47 |

TABLE 2
Results at a 30 mL/min N₂

| Reaction Time(h) | 0 | 1 | 2 | 3 | 4 | 5 |
|---|---|---|---|---|---|---|
| Peak area of solvent(%) | 91.47 | 92.33 | 92.47 | 92.36 | 92.36 | 92.35 |
| Peak area of p- | 0.00 | 0.26 | 0.27 | 0.26 | 0.26 | 0.27 |
| chloroacetophenone(%) | | | | | | |
| Peak area of p- | 0.00 | 0.43 | 0.48 | 0.47 | 0.44 | 0.42 |
| chloropropiophenone(%) | | | | | | |
| Peak area of raw material(%) | 4.65 | 3.36 | 3.25 | 3.27 | 3.33 | 3.32 |

TABLE 3
Results at a 50 mL/min N₂

| Reaction Time(h) | 0 | 1 | 2 | 3 | 4 | 5 |
|---|---|---|---|---|---|---|
| Peak area of solvent(%) | 91.47 | 91.52 | 91.52 | 91.53 | 91.47 | 91.48 |
| Peak area of p- | 0.00 | 0.24 | 0.25 | 0.23 | 0.24 | 0.24 |
| chloroacetophenone(%) | | | | | | |
| Peak area of p- | 0.00 | 0.19 | 0.20 | 0.19 | 0.18 | 0.18 |
| chloropropiophenone(%) | | | | | | |
| Peak area of raw material(%) | 4.65 | 3.88 | 4.13 | 4.08 | 4.11 | 4.14 |

TABLE 4
Results at a 70 mL/min N₂

| Reaction Time(h) | 0 | 1 | 2 | 3 | 4 | 5 |
|---|---|---|---|---|---|---|
| Peak area of solvent(%) | 91.47 | 91.53 | 91.48 | 91.49 | 91.45 | 91.48 |
| Peak area of p- | 0.00 | 0.25 | 0.25 | 0.24 | 0.24 | 0.24 |
| chloroacetophenone(%) | | | | | | |
| Peak area of p- | 0.00 | 0.18 | 0.19 | 0.19 | 0.18 | 0.19 |
| chloropropiophenone(%) | | | | | | |
| Peak area of raw material(%) | 4.65 | 3.96 | 4.04 | 4.15 | 4.13 | 4.09 |

In this blank example, after introducing nitrogen gas as the carrier gas, the peak area of the raw material increased significantly after the raw material flowed through the reaction tube. It shows that the carrier gas can significantly reduce the loss of raw materials by accelerating the flow of reaction raw materials in the reaction tube. By comparing the data at 10 mL/min, 30 mL/min and 50 mL/min nitrogen flow rates, it is seen that as the nitrogen flow rate increases, the peak areas of p-chloropropiophenone and p-chloroacetophenone (which are the by-products produced by pyrolysis of the raw material) gradually decrease, and the peak areas of raw material gradually increases. It also shows that there is no significant change in the peak area of either by-products or raw material comparing the data at a nitrogen flow rate of 50 mL/min and 70 mL/min.

### Example 2

As shown in Table 4, different catalysts were calcination modified, or both calcination-modified and steam modified as shown in “catalyst” column according to the General Treatment Procedures for Catalyst. As shown in “catalyst” column, all the temperature along with H₂O refers to the temperature for steam modification, and all the catalysts with steam modification have been modified by calcine in air at a temperature of 550° C. before the steam modification. For example, “HZSM-5-500° C.-H2O” refers to a HZSM-5 catalyst, which was both modified by calcine in air at a temperature of 550° C., and also steam modified at 500° C. according to the General Treatment Procedures for Catalyst. As shown in “catalyst” column, all the temperature without the presence of H2O refers to the temperature for calcine 15 modification. For example, “HZSM-5(25)−800° C.” refers to a HZSM-5 catalyst which was modified by calcine in air at a temperature of 800° C. according to the General Treatment Procedures for Catalyst without steam modification. Further, all the catalysts without mentioning any temperatures have been modified by calcine in air at a temperature of 550° C. For example, “HY” refers to a HY catalyst which was 20 modified by calcine in air at a temperature of 550° C. according to the General Treatment Procedures for Catalyst without steam modification. As shown in “catalyst” column, the number in the brackets refers to the Si/Al ratio of the catalysts.

The reaction reactor system as shown in FIG. 1 was loaded with catalysts or the modified catalysts. The 5-chloro-2,3-dihydro-1H-inden-1-one was prepared according to General Procedures for Preparing 5-chloro-2,3-dihydro-1H-inden-1-one. A 500 g of 10% by weight 3-chloro-1-(4-chlorophenyl)-1-propanone solution 2 in 1,2,3,4-tetrahydronaphthalene (THN) was introduced continuously into the tube reactor while nitrogen gas was pumped continuously into the tube at the same time. The Si/Al ratio of the catalysts, the reaction temperature, the amount of catalyst, the amount of DCP, the flow rate of the raw material, the GHSV of nitrogen gas were shown in Table 4. The yield of 5-chloro-2,3-dihydro-1H-inden-1-one was also shown in Table 4.

TABLE 4

| | | | | Amount | Amount | Flow | | GHSV of | |
|---|---|---|---|---|---|---|---|---|---|
| | | Si/Al | | of Cat. | of DCP | rate | T | nitrogen | Yield |
| No. | Catalyst | ratio | Solvent | (g) | (wt %) | (g/h) | (° C.) | (mL/min) | (%) |
| 1 | HY | 5 | THN | 4 | 10 | 10 | 360 | 0 | 0.00 |
| 2 | HB | 25 | THN | 4 | 10 | 10 | 360 | 0 | 0.00 |
| 3 | MCM-41 | | THN | 4 | 10 | 10 | 360 | 0 | 0.00 |
| 4 | HZSM-5 | 36 | THN | 4 | 10 | 10 | 360 | 0 | 39.76 |
| 5 | HZSM-5 | 60 | THN | 4 | 10 | 10 | 360 | 0 | 27.35 |
| 6 | HZSM-5 | 130 | THN | 4 | 10 | 10 | 360 | 0 | 25.13 |
| 7 | HZSM-5 | 200 | THN | 4 | 10 | 10 | 360 | 0 | 22.64 |
| 8 | HZSM-5-500° C.-H₂O | 36 | THN | 4 | 10 | 10 | 360 | 0 | 33.35 |
| 9 | HZSM-5-600° C.-H₂O | 36 | THN | 2 | 10 | 10 | 360 | 0 | 17.48 |
| 10 | HZSM-5-700° C.-H₂O | 36 | THN | 4 | 10 | 10 | 360 | 0 | 44.86 |
| 11 | HZSM-5-700° C.-H₂O | 36 | THN | 4 | 10 | 10 | 360 | 50 | 80.62 |
| 12 | HZSM-5 | 36 | THN | 4 | 10 | 10 | 360 | 50 | 65.69 |
| 13 | HZSM-5(36)-600° C. | 36 | THN | 4 | 10 | 10 | 360 | 50 | 58.25 |
| 14 | HZSM-5(36)-700° C. | 36 | THN | 4 | 10 | 10 | 360 | 50 | 81.43 |
| 15 | HZSM-5(36)-800° C. | 36 | THN | 4 | 10 | 10 | 360 | 50 | 88.99 |
| 16 | HZSM-5(36)-900° C. | 36 | THN | 4 | 10 | 10 | 360 | 50 | 90.64 |
| 17 | HZSM-5(36)-600° C.- | 36 | THN | 4 | 10 | 10 | 360 | 50 | 81.21 |
| | H₂O | | | | | | | | |
| 18 | HZSM-5(36)-800° C.- | 36 | THN | 4 | 10 | 10 | 360 | 50 | 86.95 |
| | H₂O | | | | | | | | |
| 19 | HZSM-5(36)-900° C.- | 36 | THN | 4 | 10 | 10 | 360 | 50 | 86.78 |
| | H₂O | | | | | | | | |
| 20 | HZSM-5(25) | 25 | THN | 4 | 10 | 10 | 360 | 50 | 63.41 |
| 21 | HZSM-5(25)-800° C. | 26 | THN | 4 | 10 | 10 | 360 | 50 | 87.28 |
| 22 | HZSM-5(25)-700° C.- | 25 | THN | 4 | 10 | 10 | 360 | 50 | 87.06 |
| | H₂O | | | | | | | | |
| 23 | HZSM-5(25)-800° C.- | 25 | THN | 4 | 10 | 10 | 360 | 50 | 90.16 |
| | H₂O | | | | | | | | |
| 24 | HZSM-5(60) | 60 | THN | 4 | 10 | 10 | 360 | 50 | 70.84 |
| 25 | HZSM-5(60)-700° C.- | 60 | THN | 4 | 10 | 10 | 360 | 50 | 85.26 |
| | H₂O | | | | | | | | |
| 26 | HZSM-5(60)-800° C. | 60 | THN | 4 | 10 | 10 | 360 | 50 | 89.81 |
| 27 | HZSM-5(130)-800° C. | 130 | THN | 4 | 10 | 10 | 360 | 50 | 83.43 |
| 28 | HZSM-5(200)-800° C. | 200 | THN | 4 | 10 | 10 | 360 | 50 | 86.72 |
| 29 | HZSM-5(200) | 200 | THN | 4 | 10 | 10 | 360 | 50 | 84.44 |
| 30 | HZSM-5(130) | 130 | THN | 4 | 10 | 10 | 360 | 50 | 81.20 |
| 31 | HZSM-5(80) | 80 | THN | 4 | 10 | 10 | 360 | 50 | 81.62 |
| 32 | Blank | | THN | 4 | 10 | 10 | 360 | 50 | 0.00 |

THN = 1,2,3,4-tetrahydronaphthalene
DCP = 3-chloro-1-(4-chlorophenyl)-1-propanone

### Example 3

As shown in Table 5, HZSM-5 catalysts dopped with different elements purchased from J&K Scientific were calcination modified, or both calcination-modified and steam modified according to the General Treatment Procedures for Catalyst. As shown in Table 5, for example, “5% P[(NH4)₂HPO₃]” means that the catalyst was dopped with 5% P in a (NH4)₂HPO₃ form. As shown in “catalyst” column, all the temperature along with H₂O refers to the temperature for steam modification, and all the catalysts with steam modification have been modified by calcine in air at a temperature of 550° C. before the steam modification. For example, “1% Mg[Mg(NO₃)₂]-HZSM-5-700° C.-H₂O” refers to a HZSM-5 catalyst which was dopped with 1% Mg in a Mg(NO₃)₂ form, was both modified by calcine in air at a temperature of 550° C., and also steam modified at 700° C. according to the General Treatment Procedures for Catalyst. As shown in “catalyst” column, all the catalysts without mentioning any temperatures have been modified by calcine in air at a temperature of 550° C. For example, “1% Zn[Zn(NO₃)₂]-HZSM-5” refers to a HZSM-catalyst which was dopped with 1% Zn in a Zn(NO₃)₂] form and was modified by calcine in air at a temperature of 550° C. according to the General Treatment Procedures for Catalyst without steam modification. As shown in “catalyst” column, the number in the brackets refers to the Si/Al ratio of the catalysts.

The reaction reactor system as shown in FIG. 1 was loaded with catalysts or the modified catalysts. The 5-chloro-2,3-dihydro-1H-inden-1-one was prepared according to General Procedures for Preparing 5-chloro-2,3-dihydro-1H-inden-1-one. A 500 g of 10% by weight 3-chloro-1-(4-chlorophenyl)-1-propanone solution 2 in 1,2,3,4-tetrahydronaphthalene (THN) was introduced continuously into the tube reactor while nitrogen gas was pumped continuously into the tube at the same time. The Si/Al ratio of the catalysts, the reaction temperature, the amount of catalyst, the amount of DCP, the flow rate of the raw material, the GHSV of nitrogen gas were shown in Table 5. The yield of 5-chloro-2,3-dihydro-1H-inden-1-one was also shown in Table 5.

TABLE 5

| | | | | Amount | Amount | Flow | | GHSV of | |
|---|---|---|---|---|---|---|---|---|---|
| | | Si/Al | | of Cat. | of DCP | rate | T | nitrogen | Yield |
| | Catalyst | ratio | Solvent | (g) | (wt %) | (g/h) | (° C.) | (mL/min) | (%) |
| 1 | 5% P[(NH₄)₂HPO₃]- | 36 | THN | 4 | 10 | 10 | 360 | 50 | 0.00 |
| | HZSM-5(powder) | | | | | | | | |
| 2 | 5% P[(NH₄)₂HPO₃]- | 36 | THN | 4 | 10 | 10 | 360 | 50 | 67.43 |
| | HZSM-5(particle) | | | | | | | | |
| 3 | 3% P[(NH₄)₂HPO₃]- | 36 | THN | 4 | 10 | 10 | 360 | 50 | 65.34 |
| | HZSM-5(particle) | | | | | | | | |
| 4 | 1% P[(NH₄)₂HPO₃]- | 36 | THN | 4 | 10 | 10 | 360 | 50 | 64.75 |
| | HZSM-5(particle) | | | | | | | | |
| 5 | 5% P[NH₄H₂PO₃]- | 36 | THN | 4 | 10 | 10 | 360 | 50 | 66.56 |
| | HZSM-5(particle) | | | | | | | | |
| 6 | 5% P(H₃PO₃)- | 36 | THN | 4 | 10 | 10 | 360 | 50 | 66.42 |
| | HZSM-5(particle) | | | | | | | | |
| 7 | 5% P[(NH₄)₂ HPO₃]- | 36 | THN | 4 | 10 | 10 | 360 | 50 | 57.27 |
| | HZSM-5-700° C.- | | | | | | | | |
| | H₂O(powder) | | | | | | | | |
| 8 | 1% B(H₃BO₃)- | 36 | THN | 4 | 10 | 10 | 360 | 50 | 68.91 |
| | HZSM-5 | | | | | | | | |
| 9 | 3% B(H₃BO₃)- | 36 | THN | 4 | 10 | 10 | 360 | 50 | 9.24 |
| | HZSM-5 | | | | | | | | |
| 10 | 1% B(H₃BO₃)- | 36 | THN | 4 | 10 | 10 | 360 | 50 | 85.35 |
| | HZSM-5-700° C.- | | | | | | | | |
| | H₂O | | | | | | | | |
| 11 | 3% B(H₃BO₃)- | 36 | THN | 4 | 10 | 10 | 360 | 50 | 69.66 |
| | HZSM-5-700° C.- | | | | | | | | |
| | H₂O | | | | | | | | |
| 12 | 1% Zn[Zn(NO₃)₂]- | 36 | THN | 4 | 10 | 10 | 360 | 50 | 63.84 |
| | HZSM-5 | | | | | | | | |
| 13 | 1% Zn[Zn(NO₃)₂]- | 36 | THN | 4 | 10 | 10 | 360 | 50 | 83.23 |
| | HZSM-5-700° C.- | | | | | | | | |
| | H₂O | | | | | | | | |
| 14 | 1% Mg[Mg(NO₃)₂]- | 36 | THN | 4 | 10 | 10 | 360 | 50 | 87.71 |
| | HZSM-5-700° C.- | | | | | | | | |
| | H₂O | | | | | | | | |
| 15 | 5% Ni[Ni(NO₃)₂]- | 200 | THN | 4 | 10 | 10 | 360 | 50 | 84.11 |
| | HZSM-5(200) | | | | | | | | |
| 16 | 5% Zn[Zn(NO₃)₂]- | 200 | THN | 4 | 10 | 10 | 360 | 50 | 71.90 |
| | HZSM-5(200) | | | | | | | | |
| 17 | 5% Zn(ZnCl₂)- | 200 | THN | 4 | 10 | 10 | 360 | 50 | 84.54 |
| | HZSM-5(200) | | | | | | | | |
| 18 | 5% Cu(CuCl₂)- | 200 | THN | 4 | 10 | 10 | 360 | 50 | 90.66 |
| | HZSM-5(200) | | | | | | | | |
| 19 | 5% Fe(FeCl₂)- | 200 | THN | 4 | 10 | 10 | 360 | 50 | 87.43 |
| | HZSM-5(200) | | | | | | | | |
| 20 | 3% Cu(CuCl₂)- | 200 | THN | 4 | 10 | 10 | 360 | 50 | 87.16 |
| | HZSM-5(200) | | | | | | | | |
| 21 | 1% Cu(CuCl₂)- | 200 | THN | 4 | 10 | 10 | 360 | 50 | 85.35 |
| | HZSM-5(200) | | | | | | | | |

THN = 1,2,3,4-tetrahydronaphthalene
DCP = 3-chloro-1-(4-chlorophenyl)-1-propanone

### Example 4

A HZSM-5 catalyst with a Si/Al ratio of 36 was both modified by calcine in air at a temperature of 550° C., and also steam modified at 800° C. according to the General Treatment Procedures for Catalyst.

The reaction reactor system as shown in FIG. 1 was loaded with the HZSM-5 catalyst modified mentioned above. The 5-chloro-2,3-dihydro-1H-inden-1-one was prepared according to General Procedures for Preparing 5-chloro-2,3-dihydro-1H-inden-1-one. A 500 g of 10% by weight 3-chloro-1-(4-chlorophenyl)-1-propanone solution 2 in 1,2,3,4-tetrahydronaphthalene (THN) was introduced continuously into the tube reactor while nitrogen gas was pumped continuously into the tube at the same time. The reaction conditions were as follows: the catalyst loading amount was 4 g, the raw material mass concentration of the feed solution was 10%, the feed rate was 10 g/h, the reaction temperature was 360° C. and the nitrogen flow rate GHSV is 12.5, 18.75, and 25 milliliter per minute per gram of catalyst, respectively.

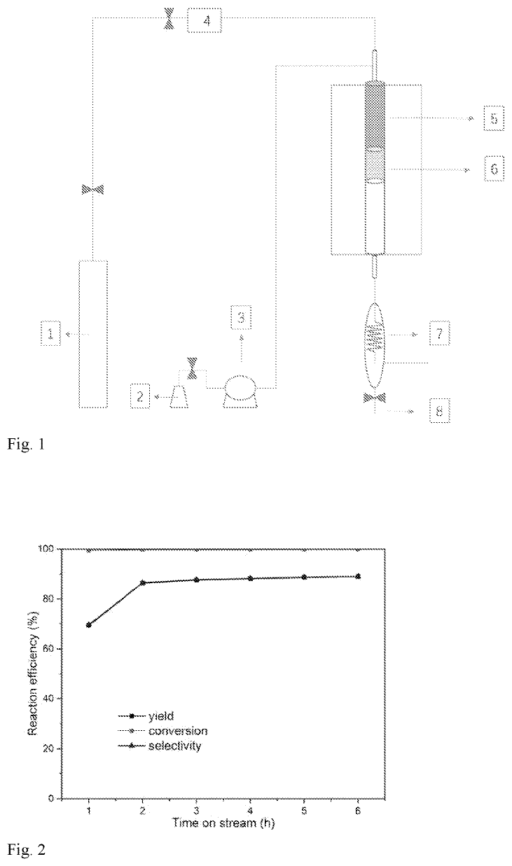

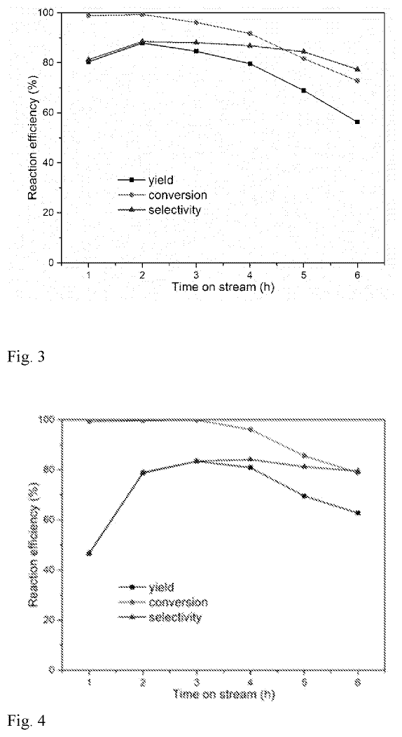

FIGS. 2 to 4 show the reaction data at a nitrogen flow rate of 12.5, 18.75, and milliliter per minute per gram of catalyst, respectively. FIG. 2 shows that at a nitrogen flow rate of 12.5 milliliter per minute per gram of catalyst, as reaction proceeded, a high conversion, a high selectivity, and a high yield were stably achieved simultaneously. It can be seen from these figures that if the nitrogen flow rate is too large, the reaction efficiency will be reduced.

All publications, patents and patent applications mentioned in this specification are herein incorporated in their entirety by reference into the specification, to the same extent as if each individual publication, patent or patent application was specifically and individually indicated to be incorporated herein by reference.

The examples illustrate the practice of the present subject matter in some of its embodiments but should not be construed as limiting the scope of the present subject matter. Other embodiments apparent to persons of ordinary skill in the art from consideration of the specification and examples herein that fall within the spirit and scope of the appended claims are part of this disclosure. The specification, including the examples, is intended to be exemplary only, without limiting the scope and spirit of the disclosure.

What is claimed is:

1. A process for preparing 5-chloro-2,3-dihydro-1H-inden-1-one comprising contacting 3-chloro-1-(4-chlorophenyl)-1-propanone continuously with a catalyst in the presence of an inert carrier gas at a gas hourly space velocity (GHSV) of 0.5 to 50 milliliter per minute per gram of catalyst.

2. The process of claim 1, wherein the GHSV of the inert carrier gas is 1 to 40 milliliter per minute per gram of catalyst.

3. The process of claim 1, wherein the GHSV of the inert carrier gas is 2 to 35 milliliter per minute per gram of catalyst.

4. The process of claim 1, wherein the GHSV of the inert carrier gas is 5 to 30 milliliter per minute per gram of catalyst.

5. The process of claim 1, wherein the GHSV of the inert carrier gas is 9 to 28 milliliter per minute per gram of catalyst.

6. The process of claim 1, wherein the GHSV of the inert carrier gas is 10 to 15 milliliter per minute per gram of catalyst.

7. The process of claim 1, wherein the catalyst is selected from the group consisting of a zeolite catalyst, MCM-41, and the combination thereof.

8. The process of claim 7, wherein the zeolite catalyst is selected from the group consisting of HY, Hβ, H-Mordenite, HZSM, and a combination thereof.

9. The process of claim 8, wherein the HZSM is HZSM-5 with a silicon to aluminum ratio of 15 to 300.

10. The process of claim 9, wherein the catalyst is HZSM-5 with a silicon to aluminum ratio of 25 to 200.

11. The process of any one of claims 8 to 10, wherein the catalyst has been modified by calcine in air at a temperature of 400 to 1000° C.

12. The process of claim 11, wherein the catalyst has been further modified by a steam.

13. The process of claim 12, wherein the steam modification is conducted in the presence of an inert carrier gas.

14. the process of claim 1 or 13, wherein the inert carrier gas is nitrogen gas.

15. The process of any one of claims 12-14, wherein the steam modification is carried out at a temperature of 400 to 900° C.

16. The process of any one of claims 11 to 15, wherein the catalyst has been further modified by doping with an element selected from the group consisting of P, B, Zn, Ni, Mg, Cu, and Fe.

17. The process of any one of claims 1 to 16, wherein the 3-chloro-1-(4-chlorophenyl)-1-propanone is fed to the catalyst as a solution in an inert solvent.

18. The process of claim 17, wherein the solvent is 1,2,3,4-tetrahydronaphthalene.

19. The process of claim 17 or 18, wherein the flow rate of 3-chloro-1-(4-chlorophenyl)-1-propanone is 0.5 to 10 g per g catalyst per hour.

20. The process of any one of claims 1 to 19, wherein the contacting is carried out in a continuous flow reactor.

21. The 5-chloro-2,3-dihydro-1H-inden-1-one prepared by the process according to any one of claims 1 to 20.

22. A process for preparing Indoxacarb comprising the steps of:
1) preparing 5-chloro-2,3-dihydro-1H-inden-1-one by the process according to any one of claims 1 to 20; and
2) preparing Indoxacarb from 5-chloro-2,3-dihydro-1H-inden-1-one prepared in step 1).

23. The Indoxacarb prepared by the process according to claim 22.
